(19) United States
(12) Patent Application Publication
(10) Pub. No.: US 2023/0277160 A1
(43) Pub. Date: Sep. 7, 2023

(54) ENDOSCOPE

(71) Applicant: FUJIFILM Corporation, Tokyo (JP)

(72) Inventor: Hirotoshi KOSAWA, Kanagawa (JP)

(73) Assignee: FUJIFILM Corporation, Tokyo (JP)

(21) Appl. No.: 18/164,616

(22) Filed: Feb. 6, 2023

(30) Foreign Application Priority Data
Mar. 3, 2022 (JP) 2022-032569

(51) Int. Cl. A61B 8/00; A61B 8/12

(52) U.S. Cl. CPC A61B 8/445; A61B 8/12

(57) ABSTRACT

A screw for fixing a frame which is an internal constituent member and a screw for fixing a bendable portion constituent member are disposed at positions different from each other in a peripheral direction, and the screws are disposed close to each other in a longitudinal axis direction relative to a case where the screws are disposed at the same position in the peripheral direction and are disposed at positions where a distance in the longitudinal axis direction between the screws is shortest.

### CROSS-REFERENCE TO RELATED APPLICATION

The present application claims priority under 35 U.S.C § 119(a) to Japanese Patent Application No. 2022-032569 filed on Mar. 3, 2022, which is hereby expressly incorporated by reference, in its entirety, into the present application.

### BACKGROUND OF THE INVENTION

### 1. Field of the Invention

The present invention relates to an endoscope and particularly relates to an endoscope having a distal end rigid portion at a distal end part of an insertion part.

### 2. Description of the Related Art

An insertion part of an endoscope to be inserted into a body comprises a distal end rigid portion, a bendable portion, and a soft portion from a distal end side toward a proximal end side.

The above-described distal end rigid portion comprises an annular connecting portion which is provided on a proximal end side of the distal end rigid portion and to which a distal end bending piece of the bendable portion is connected. In the endoscope having such a configuration, a length from a proximal end of the above-described annular connecting portion (a proximal end of the distal end rigid portion) to a distal end of the distal end rigid portion in a longitudinal axis direction of the insertion part may be referred to as a “rigid length”, and a length of a part of the distal end rigid portion excluding the above-described annular connecting portion may be referred to as a “length of the distal end part”. Hereinafter, in the present specification, a description will be made by defining the length from the proximal end of the distal end rigid portion to the distal end of the distal end rigid portion in the longitudinal axis direction of the insertion part as the “rigid length” and by defining the length of the part of the distal end rigid portion excluding the above-described annular connecting portion as the “length of the distal end part”.

Meanwhile, in a case of an endoscope of which the above-described rigid length is long, a height (also referred to as an “upright height”) of the distal end of the distal end rigid portion may become high, for example, in a case where the distal end rigid portion is raised at an angle of approximately 90 degrees with respect to the soft portion through a bending operation of the bendable portion. Then, for example, there is a problem that it is difficult to observe every corner of a stomach, and in particular, a stomach corner portion cannot be observed.

As described above, in the field of the endoscope, in order to solve the above-described problem, it is desired to shorten the rigid length, and JP2014-057731A discloses an endoscope in which the rigid length is shortened.

The endoscope disclosed in JP2014-057731A comprises a distal end constituent member that constitutes a distal end part of an insertion part, and a distal end bending piece that is externally mounted on the distal end constituent member so as to surround a proximal end part of the distal end constituent member. In addition, a projecting portion provided on an inner peripheral surface of a piece of the distal end bending piece and protruding inward from the inner peripheral surface of the piece, and a recessed portion that is provided in the distal end constituent member, that accommodates the above-described projecting portion, and that has a contact surface with which a distal end surface of the accommodated projecting portion comes into contact are further provided.

### SUMMARY OF THE INVENTION

Meanwhile, in an endoscope, in order to realize multi-functionalization, a plurality of internal constituent members, such as a forceps conduit, a lens barrel, an air and water supply conduit, and a light guide conduit, are inserted and disposed inside a distal end rigid portion, and an ultrasonic transducer, in addition to the above-described internal constituent members, is further disposed in an ultrasonic endoscope. In the endoscope having such internal constituent members, the above-mentioned problem may occur because the length of the distal end part tends to be long and, in such a case, the rigid length is also long. Therefore, in the field of the endoscope, it is required to shorten the rigid length without increasing the length of the distal end part.

The present invention has been made in view of such circumstances, and an object of the present invention is to provide an endoscope capable of shortening a rigid length without increasing a length of a distal end part.

In order to achieve the above-described object, according to the present invention, there is provided an endoscope comprising: a distal end rigid portion that constitutes a distal end side of an endoscope insertion part; a bendable portion that is bendable and is consecutively provided on a proximal end side of the distal end rigid portion; an exterior constituent member that constitutes an exterior of the distal end rigid portion; an internal constituent member disposed inside the exterior constituent member; a bendable portion constituent member that constitutes a connecting part with the exterior constituent member; a first fixture that fixes the exterior constituent member and the internal constituent member to each other; and a second fixture that fixes the exterior constituent member and the bendable portion constituent member to each other, in which the first fixture and the second fixture are disposed at positions different from each other in a peripheral direction around a longitudinal axis direction of the endoscope insertion part, and the first fixture and the second fixture are disposed close to each other in the longitudinal axis direction relative to a case where the first fixture and the second fixture are disposed at the same position in the peripheral direction and are disposed at positions where a distance in the longitudinal axis direction between the first fixture and the second fixture is shortest.

According to an aspect of the present invention, it is preferable that the exterior constituent member has a first hole portion into which the first fixture is inserted and a second hole portion into which the second fixture is inserted, the first hole portion and the second hole portion are disposed at positions different from each other in the peripheral direction, and the first hole portion and the second hole portion are disposed close to each other in the longitudinal axis direction relative to a case where the first hole portion and the second hole portion are disposed at the same position in the peripheral direction and are disposed at positions where a distance in the longitudinal axis direction between the first hole portion and the second hole portion is shortest.

According to an aspect of the present invention, it is preferable that the exterior constituent member has a hole forming portion that forms one of the first hole portion and the second hole portion, and at least a part of the hole forming portion is disposed at a position that overlaps with the other of the first hole portion and the second hole portion in the longitudinal axis direction.

According to an aspect of the present invention, it is preferable that the first hole portion and the second hole portion are disposed at positions that at least partially overlap with each other in the longitudinal axis direction.

According to an aspect of the present invention, it is preferable that a distance in the longitudinal axis direction between centers of the first hole portion and the second hole portion is shorter than a sum of a radius of the first hole portion and a radius of the second hole portion.

According to an aspect of the present invention, it is preferable that the exterior constituent member is provided with two first hole portions and two second hole portions.

According to an aspect of the present invention, it is preferable that the exterior constituent member is provided with three first hole portions and three second hole portions.

According to an aspect of the present invention, it is preferable that the first hole portion and the second hole portion are screw holes.

According to an aspect of the present invention, it is preferable that the first fixture and the second fixture are screw members.

According to an aspect of the present invention, it is preferable that the internal constituent member is an ultrasound oscillator fixing frame having an outer peripheral surface on which a plurality of ultrasound oscillators are arranged along the peripheral direction.

According to the present invention, it is possible to shorten the rigid length without increasing the length of the distal end part.

### BRIEF DESCRIPTION OF THE DRAWINGS

FIG. 1 is a schematic configuration diagram showing an example of an ultrasonography system using an ultrasonic endoscope.

FIG. 2 is an enlarged perspective view of a distal end rigid portion shown in FIG. 1.

FIG. 3 is a cross sectional view of the distal end rigid portion shown in FIG. 2.

FIG. 4 is a cross sectional view of a balloon mounted on the distal end rigid portion.

FIG. 5 is a perspective view of the distal end rigid portion including a distal end bending piece.

FIG. 6 is a side view of the distal end rigid portion shown in FIG. 5.

FIG. 7 is an enlarged view of a connecting part between the distal end rigid portion and the distal end bending piece.

FIG. 8 is a cross sectional view of an annular connecting portion in which a frame is fixed by three screws.

FIG. 9 is a cross sectional view of the annular connecting portion in which the frame is fixed by two screws.

FIG. 10 is a cross sectional view of an annular connecting portion in which the distal end bending piece is fixed by three screws.

FIG. 11 is a cross sectional view of the annular connecting portion in which the distal end bending piece is fixed by two screws.

FIGS. 12A and 12B are schematic views showing distal end rigid portions of a first embodiment and of a comparative example.

FIG. 13 is a schematic view showing a distal end rigid portion of a second embodiment.

FIG. 14 is a schematic view showing a distal end rigid portion of a third embodiment.

FIG. 15 is a schematic view showing a distal end rigid portion of a fourth embodiment.

### DESCRIPTION OF THE PREFERRED EMBODIMENTS

Hereinafter, embodiments of an endoscope according to the present invention will be described with reference to the accompanying drawings.

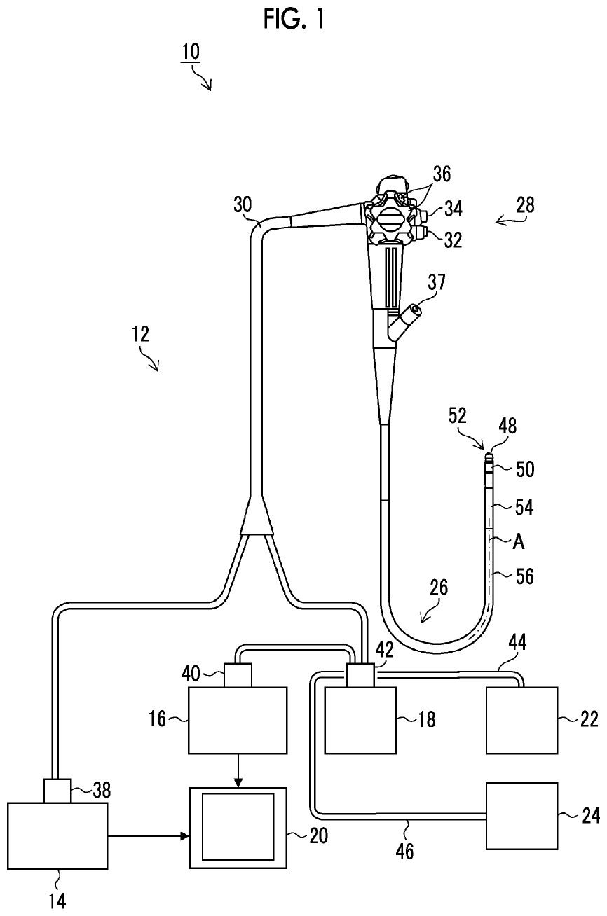

FIG. 1 is a schematic configuration diagram showing an example of an ultrasonography system 10 using an ultrasonic endoscope 12. Hereinafter, the ultrasonic endoscope 12 will be described as an example of the endoscope of the embodiment of the present invention.

As shown in FIG. 1, the ultrasonography system 10 comprises the ultrasonic endoscope 12, an ultrasonic processor device 14, an endoscope processor device 16, a light source device 18, and a monitor 20. In addition, the ultrasonography system 10 comprises a water supply tank 22 that stores wash water and the like, and a suction pump 24 that sucks a suction substance in a body cavity.

The ultrasonic endoscope 12 has an insertion part 26 to be inserted into the body cavity of a subject, an operation part 28 that is consecutively provided at a proximal end part of the insertion part 26 and that is used for an operator to perform an operation, and a universal cord 30 of which one end is connected to the operation part 28. The insertion part 26 is an example of an endoscope insertion part of the embodiment of the present invention.

The operation part 28 has an air and water supply button 32 for opening and closing an air and water supply conduit (not shown) from the water supply tank 22 and a suction button 34 for opening and closing a suction pipe line (not shown) from the suction pump 24, which are provided side by side. Further, the operation part 28 is provided with a pair of angle knobs 36 and a treatment tool insertion port 37.

The other end portion of the universal cord 30 is provided with a connector 38 connected to the ultrasonic processor device 14, a connector 40 connected to the endoscope processor device 16, and a connector 42 connected to the light source device 18. The ultrasonic endoscope 12 is attachably and detachably connected to the ultrasonic processor device 14, the endoscope processor device 16, and the light source device 18 via these connectors 38, 40, and 42. Further, the connector 42 has an air and water supply tube 44 connected to the water supply tank 22 and a suction tube 46 connected to the suction pump 24.

The insertion part 26 has, in order from a distal end side, a distal end rigid portion 52 having an endoscope observation portion 48 and an ultrasonic transducer 50, a bendable portion 54 consecutively provided on a proximal end side of the distal end rigid portion 52, and a soft portion 56 that interconnects a proximal end side of the bendable portion 54 and a distal end side of the operation part 28. The distal end rigid portion 52 constitutes the distal end side of the insertion part 26 and is an example of a distal end rigid portion of the embodiment of the present invention. The distal end rigid portion 52, the bendable portion 54, and the soft portion 56 are disposed along a longitudinal axis A of the elongated insertion part 26. The bendable portion 54 is formed by connecting a plurality of bending pieces so as to be bendable. The soft portion 56 is thin and elongated, and has flexibility.

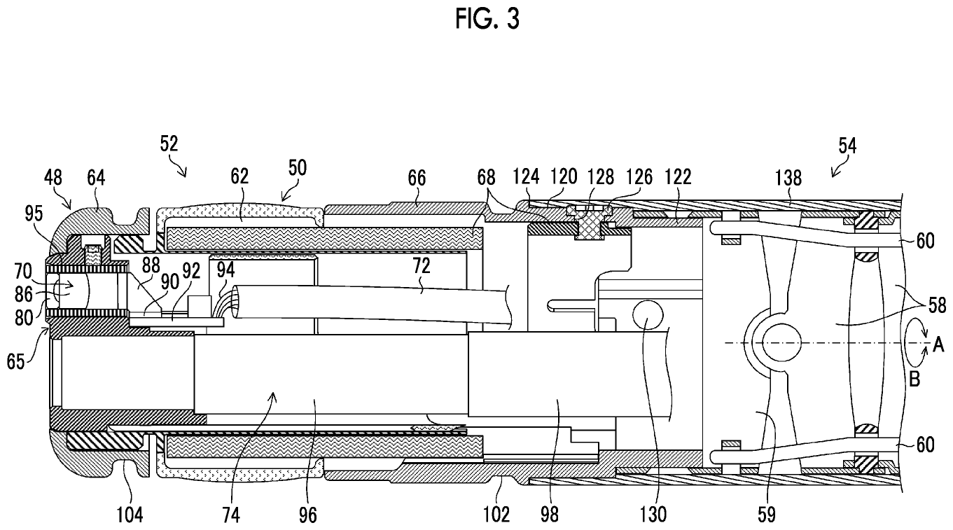

The bendable portion 54 is remotely operated to be bent through a rotationally moving operation of the pair of angle knobs 36 provided in the operation part 28. With this, the distal end rigid portion 52 can be directed in a desired direction. Further, FIG. 3, which will be described later, shows a plurality of bending pieces 58 constituting the bendable portion 54, and a plurality of bending operation wires 60 (two in FIG. 3). Among the plurality of bending pieces 58, a distal end bending piece 59 disposed on a distal end side is connected to an annular connecting portion 122 formed on the proximal end side of the distal end rigid portion 52. A distal end side of the bending operation wire 60 is connected to the distal end bending piece 59, and a proximal end side of the bending operation wire 60 is connected to the pair of angle knobs 36 (see FIG. 1). The above-described annular connecting portion 122 will be described later. In addition, a connecting structure for connecting the distal end bending piece 59 and the annular connecting portion 122 to each other will also be described later.

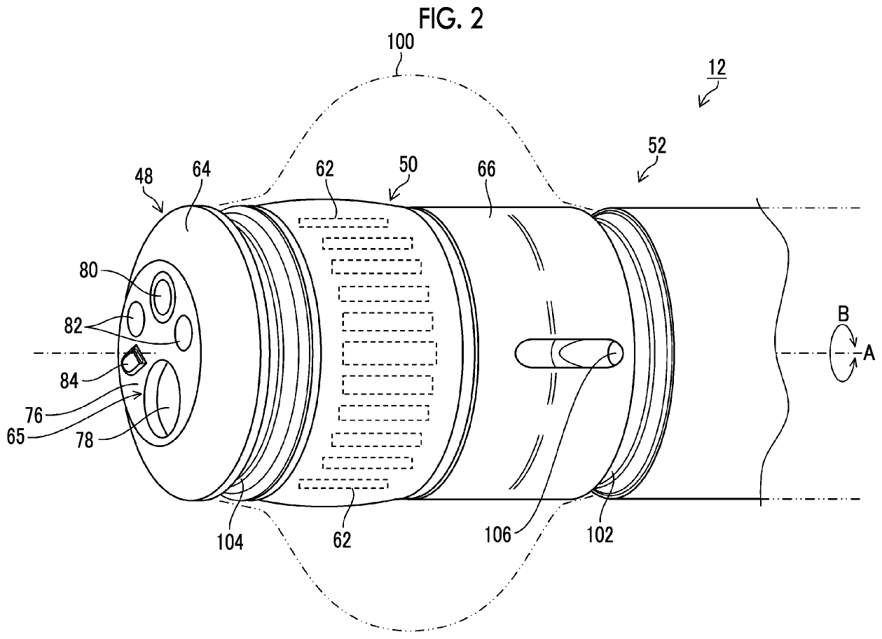

Returning to FIG. 1, the ultrasonic processor device 14 generates and supplies an ultrasonic signal for generating an ultrasonic wave to a plurality of ultrasound oscillators 62 (see FIG. 2) constituting the ultrasonic transducer 50. The ultrasonic wave is radiated from the plurality of ultrasound oscillators 62 toward a part to be observed. The ultrasonic processor device 14 receives and acquires an echo signal (reflected wave) reflected from the part to be observed through the ultrasound oscillators 62, and performs various types of signal processing on the acquired echo signal to generate an ultrasonic image. The generated ultrasonic image is displayed on the monitor 20.

In addition, the part to be observed is illuminated with illumination light emitted from the light source device 18, in the endoscope observation portion 48. The endoscope processor device 16 receives and acquires an image signal acquired from the part to be observed, and performs various types of signal processing and image processing on the acquired image signal to generate an endoscopic image. The generated endoscopic image is displayed on the monitor 20.

The monitor 20 receives each video signal generated by the ultrasonic processor device 14 and the endoscope processor device 16 and displays the ultrasonic image and the endoscopic image. In the display of the ultrasonic image and the endoscopic image, it is also possible to display only one image on the monitor 20 by appropriately switching therebetween or to display both the images at the same time.

Next, the configuration of the distal end rigid portion 52 will be described with reference to FIGS. 2 and 3. FIG. 2 is an enlarged perspective view of the distal end rigid portion 52 shown in FIG. 1. FIG. 3 is a cross sectional view of the distal end rigid portion 52 shown in FIG. 2.

As shown in FIG. 2, the distal end rigid portion 52 has the endoscope observation portion 48 for acquiring the endoscopic image provided on a distal end side, and has the ultrasonic transducer 50 for acquiring the ultrasonic image provided on the proximal end side.

As shown in FIGS. 2 and 3, the distal end rigid portion 52 has an annular distal end side cap 64 disposed on the distal end side with respect to the ultrasonic transducer 50 and a tubular exterior constituent member 66 disposed on the proximal end side with respect to the ultrasonic transducer 50. The distal end side cap 64 is mounted on a distal end part body 65 to constitute a distal end of the distal end rigid portion 52. The distal end side cap 64, the distal end part body 65, and the exterior constituent member 66 each consist of an insulating member, such as a rigid resin. The exterior constituent member 66 constitutes an exterior of the distal end rigid portion 52 and is an example of an exterior constituent member of the embodiment of the present invention.

As shown in FIG. 3, a tubular frame 68 is connected to a proximal end side of the distal end side cap 64. An observation system unit 70, a shielded cable 72, a forceps conduit 74, and the like, which will be described later, are disposed inside the frame 68, and the ultrasonic transducer 50 is disposed outside the frame 68. The frame 68 is made of, for example, metal having high material strength in order to support the ultrasonic transducer 50. With the frame 68 made of metal, the frame 68 can function as an electromagnetic wave shielding member. The frame 68 is disposed inside the exterior constituent member 66 and is an example of an internal constituent member of the embodiment of the present invention.

As shown in FIG. 2, the ultrasonic transducer 50 is configured by arranging the plurality of ultrasound oscillators 62, which transmit and receive ultrasonic waves, in a circumferential direction of an outer peripheral wall of the frame 68 (see FIG. 3). That is, the ultrasonic transducer 50 of this example is a radial type ultrasonic transducer in which the plurality of ultrasound oscillators 62 are arranged along a peripheral direction around the longitudinal axis A.

The plurality of ultrasound oscillators 62 are connected to a plurality of cables (not shown), respectively. The plurality of cables are inserted into the operation part 28 from the bendable portion 54 shown in FIG. 1 via the soft portion 56, for example, in a state of being accommodated in an ultrasonic shielded cable. Then, the plurality of cables are inserted from the operation part 28 into the universal cord 30 and are connected to the ultrasonic connector 38. The connector 38 is connected to the ultrasonic processor device 14. The ultrasonic signal generated by the ultrasonic processor device 14 is supplied to the plurality of ultrasound oscillators 62 via the plurality of cables.

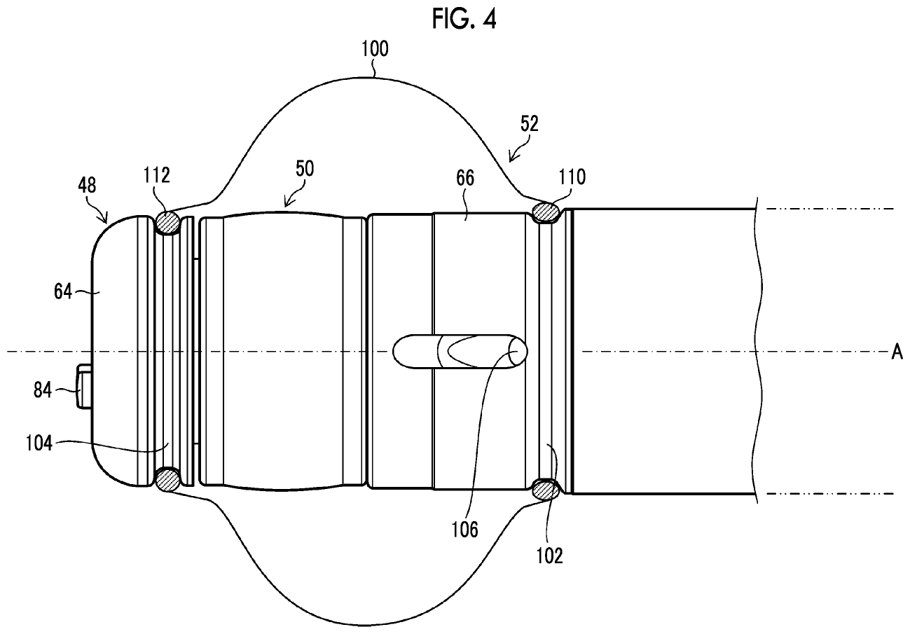

As shown in FIG. 2, a balloon 100 (see FIG. 4) surrounding the ultrasonic transducer 50 is attachably and detachably mounted on the distal end rigid portion 52. An ultrasonic wave transmission medium (for example, water) is supplied into the balloon 100. Here, the ultrasonic wave and the echo signal are attenuated in the air. For this reason, the balloon 100 is inflated by being supplied with water, and the inflated balloon 100 is brought into contact with the part to be observed, thereby excluding the air from between the ultrasonic transducer 50 and the part to be observed. With this, the attenuation of the ultrasonic wave and the echo signal can be restrained, so that a good ultrasonic image can be obtained. The balloon 100 will be described later.

As shown in FIG. 2, the endoscope observation portion 48 has a treatment tool outlet port 78, an observation window 80, an illumination window 82, a nozzle 84, and the like, which are open to a distal end surface 76 of the distal end part body 65. That is, the ultrasonic endoscope 12 of this example is a forward viewing type ultrasonic endoscope having the observation window 80 on the distal end surface 76 of the distal end rigid portion 52. A pair of the illumination windows 82 are provided with the observation window 80 interposed therebetween.

As shown in FIG. 3, the forceps conduit 74 is connected to the treatment tool outlet port 78. The forceps conduit 74 has a forceps pipe 96 of which a distal end side is connected to the treatment tool outlet port 78, and a forceps tube 98 of which a distal end side is connected to a proximal end side of the forceps pipe 96. The forceps tube 98 extends from an inside of the bendable portion 54 to a proximal end side of the soft portion 56 (see FIG. 1), and a proximal end side of the forceps tube 98 is connected to the treatment tool insertion port 37 of the operation part 28 (see FIG. 1). A treatment tool, such as a forceps, is inserted into the forceps tube 98 from the treatment tool insertion port 37 and is led out from the treatment tool outlet port 78 via the forceps pipe 96.

As shown in FIG. 3, the observation system unit 70 is disposed behind (on the proximal end side of) the observation window 80. The observation system unit 70 has an objective lens 86, a prism 88, an imaging element 90, a substrate 92, a signal cable 94, and the like. The objective lens 86 is provided in the distal end part body 65 in a state of being fixed to a lens barrel 95.

The reflected light of the part to be observed incident from the observation window 80 is captured by the objective lens 86. An optical path of the captured reflected light is bent at a right angle by the prism 88, and an image thereof is formed on an imaging surface of the imaging element 90. The imaging element 90 outputs an image signal by photoelectrically converting the reflected light of the part to be observed of which the image is formed on the above-described imaging surface. Examples of the imaging element 90 include a charge coupled device (CCD) and a complementary metal oxide semiconductor (CMOS).

The imaging element 90 is mounted on the substrate 92. A circuit pattern (not shown) electrically connected to the imaging element 90 is formed in the substrate 92. The circuit pattern has a plurality of electrodes, and a plurality of the signal cables 94 are connected to these electrodes, respectively. The plurality of signal cables 94 are inserted into the operation part 28 from the bendable portion 54 shown in FIG. 1 via the soft portion 56 in a state of the shielded cable 72 including the plurality of signal cables 94. Then, the plurality of signal cables 94 are inserted from the operation part 28 into the universal cord 30 and are connected to the endoscopic connector 40. The connector 40 is connected to the endoscope processor device 16.

An emission end of a light guide (not shown) is connected to each of the two illumination windows 82 shown in FIG. 2. The light guide extends from the insertion part 26 shown in FIG. 1 to the operation part 28 and is inserted from the operation part 28 into the universal cord 30, and an incidence end of the light guide is connected to the light source connector 42. The connector 42 is connected to the light source device 18. The illumination light emitted by the light source device 18 propagates through the light guide and is emitted from the illumination window 82 of FIG. 2.

A distal end side of the air and water supply conduit (not shown) is connected to the nozzle 84 shown in FIG. 2. The air and water supply conduit extends from the insertion part 26 shown in FIG. 1 to the operation part 28 and is inserted into the universal cord 30 from the operation part 28, and a proximal end side of the air and water supply conduit is connected to the light source connector 42. With this, the proximal end side of the air and water supply conduit is connected to the water supply tank 22 via the connector 42 and the air and water supply tube 44. The water in the water supply tank 22 is supplied from the air and water supply tube 44 to the air and water supply conduit via the connector 42 and is ejected from the nozzle 84 toward the observation window 80 and the illumination window 82. In addition, air supplied from an air pump (not shown) is supplied to the air and water supply conduit, and the air is ejected from the nozzle 84 toward the observation window 80 and the illumination window 82 via the air and water supply conduit.

Next, the balloon 100 shown in FIG. 4 will be described. FIG. 4 is a cross sectional view of the balloon 100 mounted on the distal end rigid portion 52.

As shown in FIG. 4, a mounting groove 102 for mounting a proximal end side of the balloon 100 is formed on an outer peripheral surface of the exterior constituent member 66, and a mounting groove 104 for mounting a distal end side of the balloon 100 is formed on an outer peripheral surface of the distal end side cap 64. These mounting grooves 102 and 104 are formed along the peripheral direction around the longitudinal axis A.

Further, a supply port 106 for supplying water into the balloon 100 and for discharging the water in the balloon 100 is formed between the mounting groove 102 and the mounting groove 104 on an outer surface of the exterior constituent member 66. Further, the supply port 106 is formed between the mounting groove 102 and the ultrasonic transducer 50.

The balloon 100 is formed of an elastic member, such as rubber. The balloon 100 has a ring-shaped band portion 110 provided on one end side of both ends of the balloon 100 and a ring-shaped band portion 112 provided on the other end side. In the balloon 100, the band portion 110 is elastically mounted on the mounting groove 102, and the band portion 112 is elastically mounted on the mounting groove 104, with respect to the distal end rigid portion 52.

Meanwhile, in the field of the endoscope, for the purpose of improving operability, it is required in the distal end rigid portion to shorten a rigid length without increasing a length of the distal end part. In that respect, the ultrasonic endoscope 12 of this example employs the following configuration in order to shorten the rigid length without increasing the length of the distal end part.

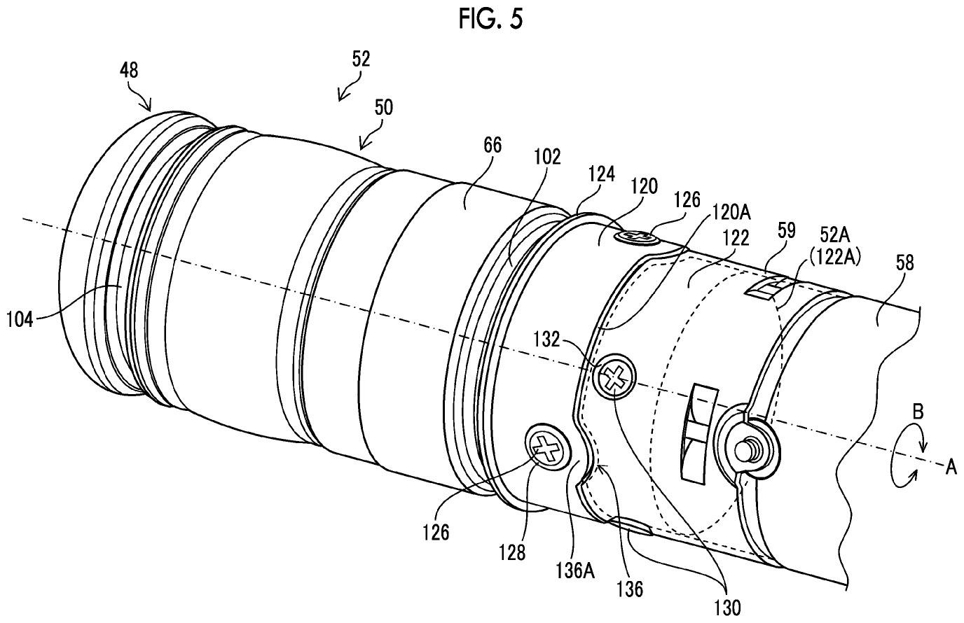

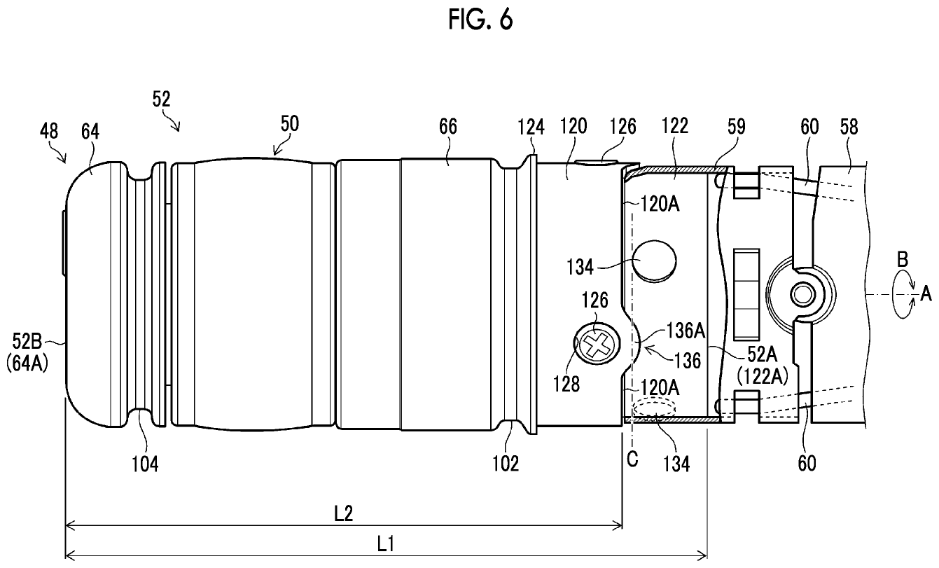

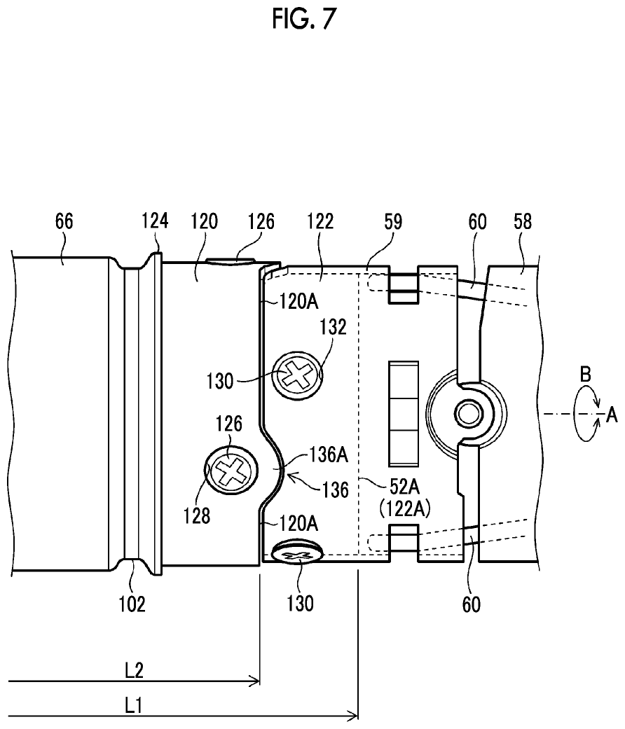

Hereinafter, the configuration of the distal end rigid portion 52 according to a first embodiment will be described with reference to FIGS. 3, 5, 6, and 7. FIG. 5 is a perspective view of the distal end rigid portion 52 including the distal end bending piece 59, and FIG. 6 is a side view of the distal end rigid portion 52 shown in FIG. 5 and is a view showing the distal end bending piece 59 partially cut away from FIG. 5. FIG. 7 is an enlarged view of a connecting part between the distal end rigid portion 52 and the distal end bending piece 59.

As shown in FIGS. 5 to 7, the distal end rigid portion 52 has the exterior constituent member 66 that constitutes the distal end rigid portion 52, and two annular connecting portions 120 and 122 are consecutively provided on a proximal end side of the exterior constituent member 66 along a direction of the longitudinal axis A. That is, the annular connecting portions 120 and 122 constitute a part of the exterior constituent member 66. The annular connecting portion 120 is provided so as to protrude from a flange portion 124 consecutively provided on the mounting groove 102 of the balloon 100 (see FIG. 4) toward the proximal end side of the insertion part 26 (see FIG. 1). The flange portion 124 is formed along a peripheral direction B around the direction of the longitudinal axis A and has a diameter larger than a diameter of the annular connecting portion 120. Further, the annular connecting portion 122 protrudes from the annular connecting portion 120 toward the proximal end side of the insertion part 26 (see FIG. 1) and is formed to have a diameter smaller than the diameter of the annular connecting portion 120.

Here, the length of the distal end part and the rigid length described above will be described with reference to FIG. 6. First, the rigid length indicates a length L1 from a proximal end 52A (a proximal end 122A of the annular connecting portion 122) of the distal end rigid portion 52 to a distal end 52B (a distal end 64A of the distal end side cap 64) of the distal end rigid portion 52 in the direction of the longitudinal axis A of the insertion part 26. In addition, the length of the distal end part indicates a length (a length of a part of the distal end rigid portion 52 excluding the annular connecting portion 122) L2 from a proximal end 120A of the annular connecting portion 120 to the distal end 52B of the distal end rigid portion 52 in the direction of the longitudinal axis A of the insertion part 26.

As shown in FIGS. 5 to 7, a screw 126 is provided in the annular connecting portion 120. The screw 126 is inserted into a hole portion 128 disposed in the annular connecting portion 120 and is fastened to the frame 68 (see FIG. 3) disposed inside the exterior constituent member 66. Therefore, the exterior constituent member 66 and the frame 68 (see FIG. 3) are fixed to each other by the screw 126. The screw 126 is an example of a first fixture of the embodiment of the present invention and is an example of a screw member. In this example, although the screw 126 is illustrated as the first fixture, the present invention is not limited to the screw 126, and another fixture, such as a rivet, may be applied as the first fixture. Further, the hole portion 128 is an example of a first hole portion of the embodiment of the present invention and is an example of a screw hole.

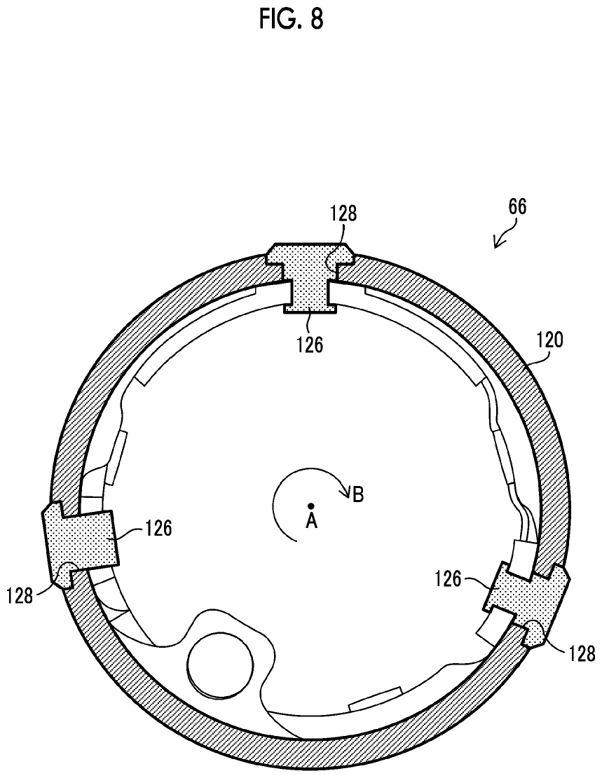

FIG. 8 is a cross sectional view of the annular connecting portion 120 (a cross sectional view in a direction orthogonal to the direction of the longitudinal axis A). As shown in FIG. 8, three hole portions 128 are provided along the peripheral direction B of the annular connecting portion 120. The three hole portions 128 shown in FIG. 8 are disposed at substantially equal intervals in the peripheral direction B. With the distal end rigid portion 52 of this example in which the exterior constituent member 66 and the frame 68 (see FIG. 3) are fixed to each other by using the three hole portions 128 as described above, the tubular frame 68 can be stably fixed to the exterior constituent member 66 inside the exterior constituent member 66.

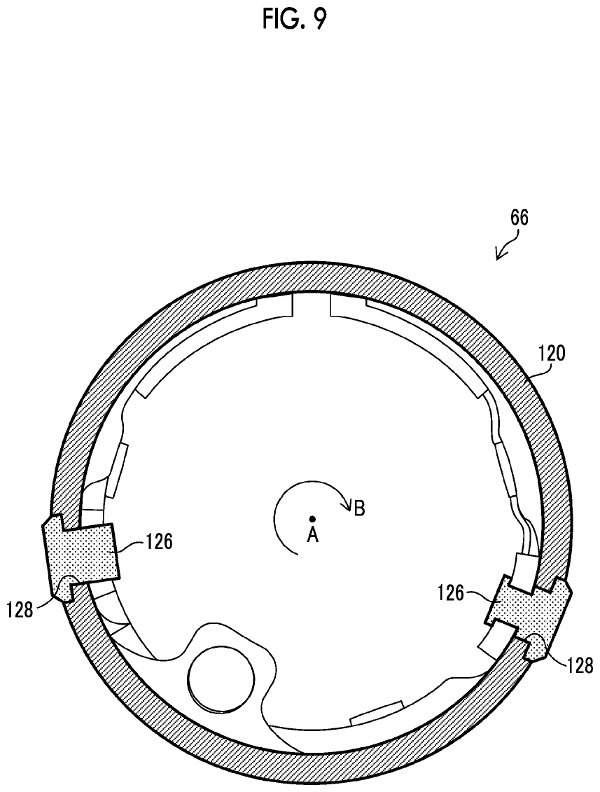

In addition, in this example, although the exterior constituent member 66 with a form in which the three hole portions 128 are disposed is illustrated, the present invention is not limited thereto, and the exterior constituent member 66 with, for example, a form in which two hole portions 128 are disposed may be employed as in the cross sectional view of the annular connecting portion 120 shown in FIG. 9. Even with this form, the frame 68 can be stably fixed to the exterior constituent member 66. However, since the frame 68 is formed in a tubular shape, the frame 68 can be fixed more stably in the form having the three hole portions 128 than in the form having the two hole portions 128. Further, the exterior constituent member 66 with a form in which one hole portion 128 is disposed may be employed. With this form, other internal constituent members other than the frame 68 (for example, other internal constituent members, such as a light guide conduit (not shown)) can be stably fixed to the exterior constituent member 66.

As shown in FIGS. 5 and 7, the annular connecting portion 122 is provided with a screw 130. The screw 130 is inserted via a hole portion 132 disposed in the distal end bending piece 59 and is fastened to a hole portion 134 (see FIG. 6) disposed in the annular connecting portion 122. Therefore, the exterior constituent member 66 and the distal end bending piece 59 are fixed to each other by the screw 130. The screw 130 is an example of a second fixture of the embodiment of the present invention and is an example of the screw member. In this example, although the screw 130 is illustrated as the second fixture, the present invention is not limited to the screw 130, and another fixture, such as a rivet, may be applied as the second fixture. Further, the hole portion 134 is an example of a second hole portion of the embodiment of the present invention and is an example of the screw hole. In addition, the distal end bending piece 59 is an example of a bendable portion constituent member of the embodiment of the present invention.

As shown in FIG. 3, in the distal end bending piece 59 of this example, a wall thickness of the distal end bending piece 59 is set to about half a difference in diameter between the annular connecting portion 120 and the annular connecting portion 122. With this, in a case where the distal end bending piece 59 is externally mounted on and connected to the annular connecting portion 122, an outer surface of the distal end bending piece 59 and an outer surface of the annular connecting portion 120 are substantially flush with each other. Further, as shown in FIG. 3, a tubular angle rubber 138 constituting an outer cover of the bendable portion 54 covers the outer surface of the distal end bending piece 59 and the outer surface of the annular connecting portion 120 which are substantially flush with each other.

FIG. 10 is a cross sectional view of the annular connecting portion 122 (a cross sectional view in the direction orthogonal to the direction of the longitudinal axis A). As shown in FIG. 10, three hole portions 134 are provided along the peripheral direction B of the annular connecting portion 122. The three hole portions 134 shown in FIG. 10 are disposed at substantially equal intervals in the peripheral direction B. With the distal end rigid portion 52 of this example in which the exterior constituent member 66 and the distal end bending piece 59 are fixed by using the three hole portions 134 as described above, the distal end bending piece 59 can be stably fixed to the exterior constituent member 66.

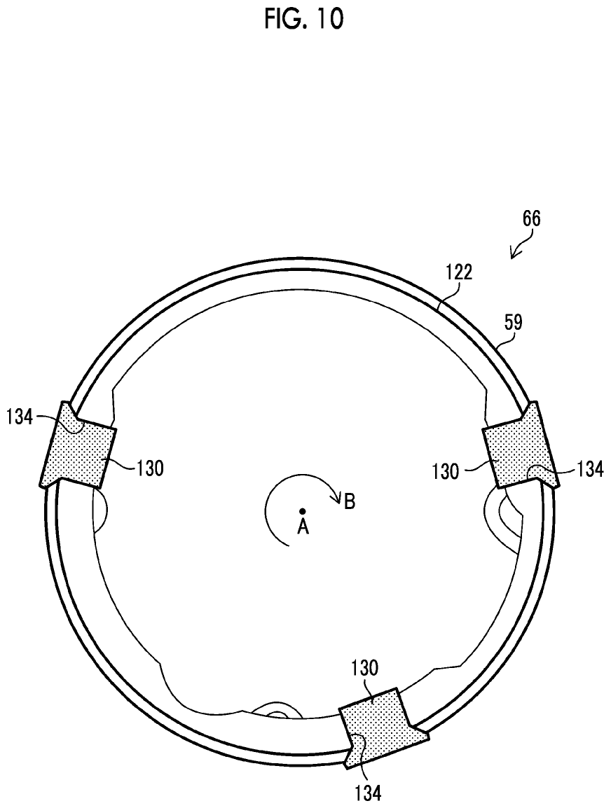

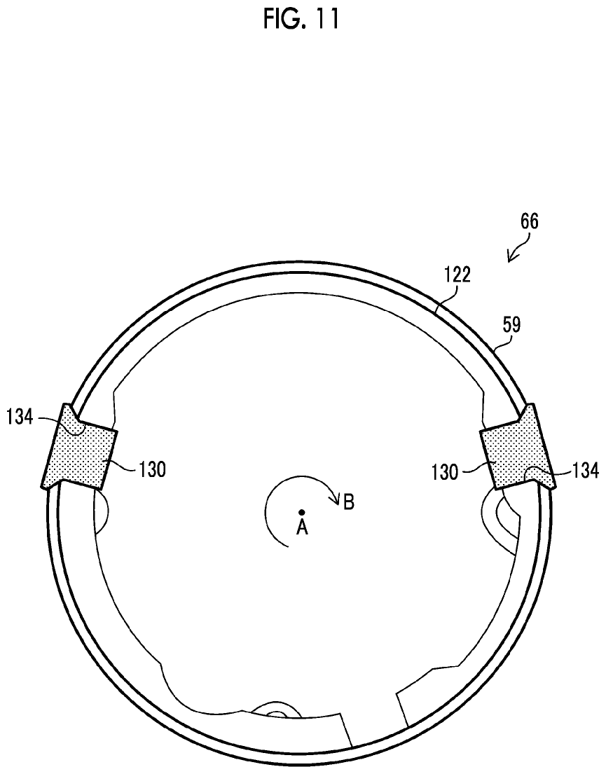

In addition, in this example, although the exterior constituent member 66 with a form in which the three hole portions 134 are disposed is illustrated, the present invention is not limited thereto, and the exterior constituent member 66 with, for example, a form in which two hole portions 134 are disposed may be employed as in the cross sectional view of the annular connecting portion 122 shown in FIG. 11. Even with this form, the distal end bending piece 59 can be stably fixed to the exterior constituent member 66. However, since the distal end bending piece 59 is formed in a tubular shape, the distal end bending piece 59 can be fixed more stably in the form having the three hole portions 134 than in the form having the two hole portions 134.

The annular connecting portion 120 will be described hereinafter. As shown in FIGS. 5 to 7, the annular connecting portion 120 has a hole forming portion 136 for forming the hole portion 128. The hole forming portion 136 is formed so as to protrude from the proximal end 120A of the annular connecting portion 120 over the annular connecting portion 122. Further, the hole forming portion 136 has a semi-circular portion 136A, and the semi-circular portion 136A is provided so as to overlap with a part of the annular connecting portion 122. Further, at least a part of the semi-circular portion 136A is disposed at a position that overlaps with the hole portion 134 (FIG. 6) in the direction of the longitudinal axis A.

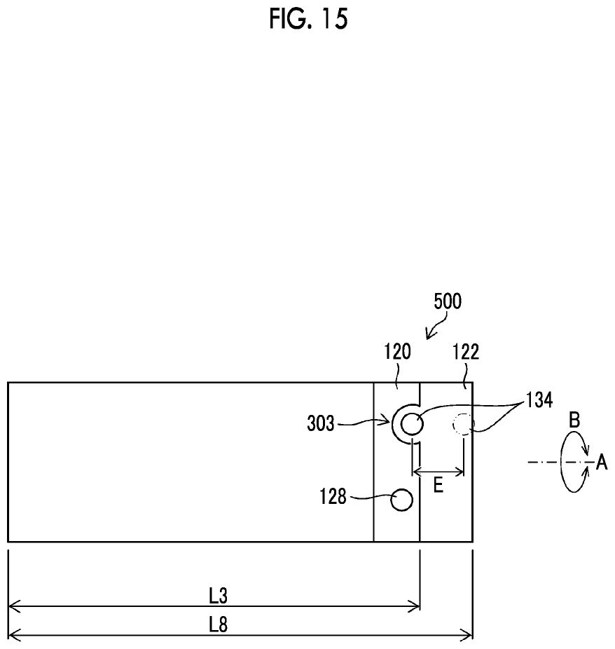

Specifically, as shown in FIG. 6, in a case where the hole forming portion 136 and the hole portion 134 are viewed from the direction orthogonal to the longitudinal axis A, at least a part of the hole forming portion 136 is disposed on the proximal end side of the insertion part 26 (see FIG. 1) with respect to a tangent line C which is a virtual line. This tangent line C is a tangent line that is located on a distal end side among tangent lines tangent to the hole portion 134 and that is directed in the direction orthogonal to the longitudinal axis A. More specifically, in a region of the semi-circular portion 136A of the hole forming portion 136, for example, a region part of about 30% to 50% is disposed on the proximal end side of the insertion part 26 (see FIG. 1) with respect to the tangent line C. A configuration having such a hole forming portion 136 is employed, whereby the hole portion 128 can be disposed near the proximal end 120A of the annular connecting portion 120. In addition, as will be described later, the hole portion 128 and the hole portion 134 can be disposed at positions that at least partially overlap with each other in the direction of the longitudinal axis A (see FIGS. 14 and 15).

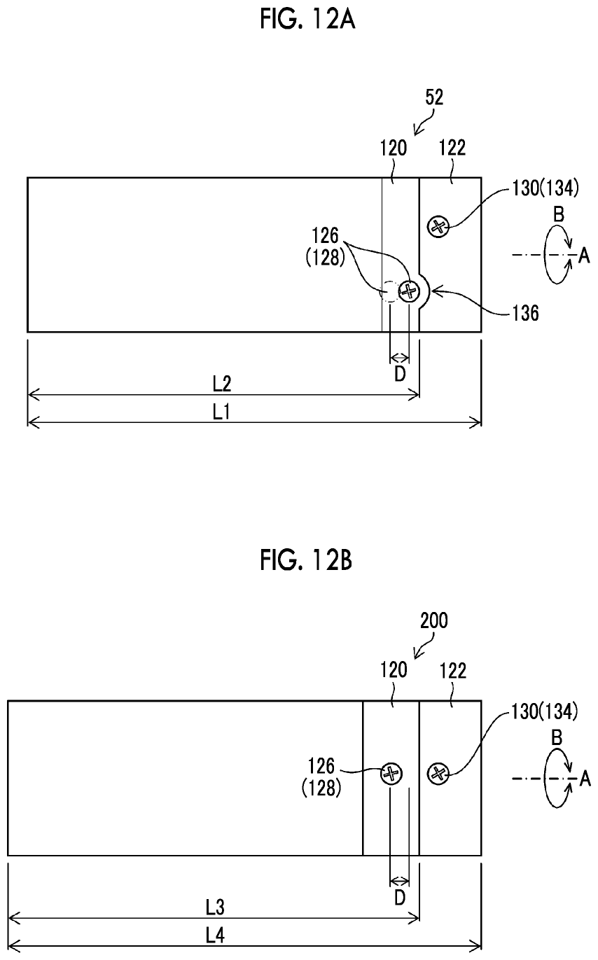

Next, specific disposition positions of the screw 126 and the screw 130 will be described with reference to FIGS. 12A and 12B.

FIGS. 12A and 12B are schematic views of distal end rigid portions 52 and 200 showing an example of the disposition positions of the screw 126 and the screw 130. FIG. 12A schematically shows the configuration of the distal end rigid portion 52 of the first embodiment, and FIG. 12B schematically shows the configuration of the distal end rigid portion 200 of a comparative example.

In the distal end rigid portion 200 shown as the comparative example, the screw 126 and the screw 130 are disposed at the same position in the peripheral direction B and are disposed at positions where a distance in the direction of the longitudinal axis A between the screw 126 and the screw 130 is the shortest.

With respect to the distal end rigid portion 200 having such a configuration, in the distal end rigid portion 52 of the first embodiment, the screw 126 and the screw 130 are disposed at positions shifted from each other in the peripheral direction B, and the screw 126 and the screw 130 are disposed close to each other in the direction of the longitudinal axis A relative to a case of the form of the above-described distal end rigid portion 200.

Specifically, with the distal end rigid portion 52 of the first embodiment, the screw 126 is disposed closer to the screw 130 by a distance D than to the screw 130 in the configuration of the distal end rigid portion 200. With this, the length L2 of the distal end part of the distal end rigid portion 52 can be shortened by the distance D from a length L3 of a distal end part of the distal end rigid portion 200, and a rigid length L1 can be shortened by the distance D from a rigid length L4 of the distal end rigid portion 200.

Accordingly, since the distal end rigid portion 52 of the first embodiment employs a configuration in which the screw 126 and the screw 130 are disposed at positions different from each other in the peripheral direction B and the screw 126 and the screw 130 are disposed close to each other in the direction of the longitudinal axis A relative to a case of the configuration of the distal end rigid portion 200 (a case where the screw 126 and the screw 130 are disposed at the same position in the peripheral direction B and are disposed at positions where the distance in the direction of the longitudinal axis A between the screw 126 and the screw 130 is the shortest), it is possible to shorten the rigid length without increasing the length of the distal end part.

In addition, in another expression, the above-described configuration can be described that the distal end rigid portion 52 of the first embodiment has a configuration in which the hole portion 128 and the hole portion 134 are disposed at positions different from each other in the peripheral direction B and the hole portion 128 and the hole portion 134 are disposed close to each other in the direction of the longitudinal axis A relative to a case (the case of the configuration of the distal end rigid portion 200) where the hole portion 128 and the hole portion 134 are disposed at the same position in the peripheral direction B and are disposed at positions where the distance in the direction of the longitudinal axis A between the hole portion 128 and the hole portion 134 is the shortest. As described above, the same effect as described above can also be obtained by specifying the positions of the hole portion 128 and the hole portion 134 in the same manner as specifying the positions of the screw 126 and the screw 130.

Further, in the distal end rigid portion 52 of the first embodiment, as shown in FIG. 6, since the hole forming portion 136 for forming the hole portion 128 in the exterior constituent member 66 is formed and at least a part of the hole forming portion 136 is disposed at a position that overlaps with the hole portion 134 in the direction of the longitudinal axis A, the hole portion 128 can be easily brought close to the hole portion 134 in the direction of the longitudinal axis A.

As shown in FIG. 12A, although the distal end rigid portion 52 of the first embodiment has a configuration in which the screw 126 (hole portion 128) is brought close to the screw 130 (hole portion 134), instead of this configuration, a configuration in which the screw 130 (hole portion 134) is brought close to the screw 126 (hole portion 128) may be employed.

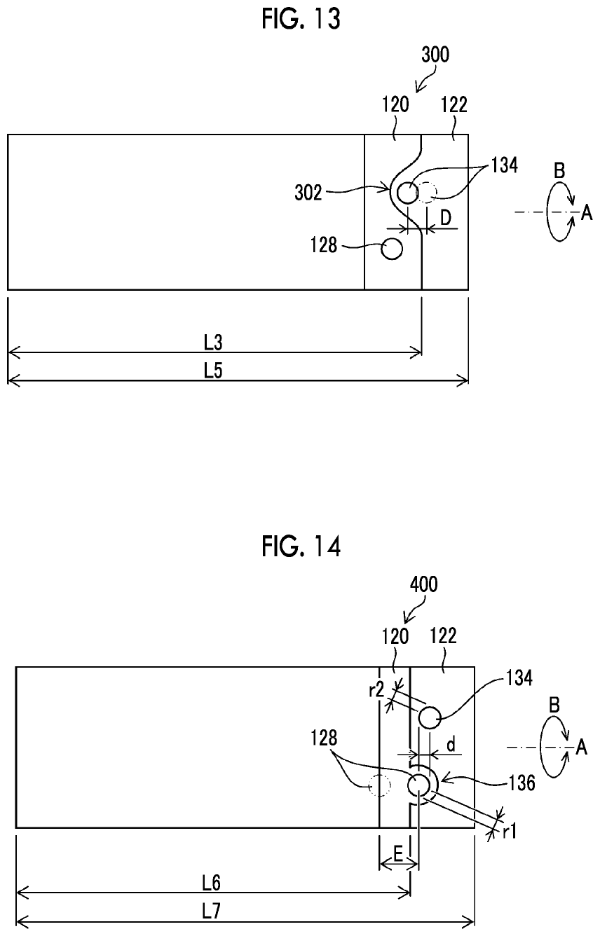

FIG. 13 is a schematic view of a distal end rigid portion 300 of a second embodiment, and a configuration is employed in which the screw 130 (hole portion 134) is brought close to the screw 126 (hole portion 128). With the distal end rigid portion 300 of the second embodiment, the screw 130 can be brought closer to the screw 126 by the distance D than to the screw 126 in the configuration of the distal end rigid portion 200 (see FIG. 12B). With this, a rigid length L5 can be shortened by the distance D from the rigid length L4 of the distal end rigid portion 200, while the length L3 of the distal end part of the distal end rigid portion 300 is maintained at the length L3 of the distal end part of the distal end rigid portion 200. As a result, it is possible to shorten the rigid length without increasing the length of the distal end part.

In addition, in the distal end rigid portion 300 of the second embodiment, as shown in FIG. 13, a hole forming portion 302 for forming the hole portion 134 in the exterior constituent member 66 is formed, and at least a part of the hole forming portion 302 is disposed at a position that overlaps with the hole portion 128 in the direction of the longitudinal axis A. With this, the hole portion 134 is easily brought close to the hole portion 128 in the direction of the longitudinal axis A. The hole forming portion 302 of this example is formed from the annular connecting portion 122 over the annular connecting portion 120, and is formed by, as an example, a recessed portion formed in the annular connecting portion 120.

FIG. 14 is a schematic view of a distal end rigid portion 400 of a third embodiment. The distal end rigid portion 400 of the third embodiment employs a configuration in which the hole portion 128 and the hole portion 134 are disposed at positions that at least partially overlap with each other in the direction of the longitudinal axis A. In other words, a configuration is employed in which a distance d in the direction of the longitudinal axis A between centers of the hole portion 128 and the hole portion 134 is shorter than a sum of a radius r1 of the hole portion 128 and a radius r2 of the hole portion 134.

Specifically, in the configuration of the distal end rigid portion 400 of the third embodiment, the hole portion 128 is disposed closer to the hole portion 134 by a distance E (E>D) than to the hole portion 134 in the configuration of the distal end rigid portion 200 shown in FIG. 12B. With this, a length L6 of the distal end part of the distal end rigid portion 400 can be shortened by the distance E from the length L3 of the distal end part of the distal end rigid portion 200, and a rigid length L7 can be shortened by the distance E from the rigid length L4 of the distal end rigid portion 200.

FIG. 15 is a schematic view of a distal end rigid portion 500 of a fourth embodiment, and a configuration is employed in which the hole portion 134 is brought close to the hole portion 128, in the configurations in which the hole portion 128 and the hole portion 134 are disposed at positions that at least partially overlap with each other in the direction of the longitudinal axis A. With the distal end rigid portion 500 of the fourth embodiment, the hole portion 134 can be brought close to the hole portion 128 by the distance E than to the hole portion 128 in the configuration of the distal end rigid portion 200 (see FIG. 12B). With this, a rigid length L8 can be shortened by the distance E from the rigid length L4 of the distal end rigid portion 200, while the length L3 of the distal end part of the distal end rigid portion 500 is maintained at the length L3 of the distal end part of the distal end rigid portion 200. As a result, it is possible to shorten the rigid length without increasing the length of the distal end part.

Others

In the above-described embodiments, the ultrasonic endoscope 12 has been described as an example of an endoscope to which the present invention is applied, but the present invention is not limited to the ultrasonic endoscope. For example, the present invention can also be applied to a general endoscope provided without ultrasonic transducer, such as a colonoscope and a small intestinal endoscope.

Although the endoscope according to the embodiment has been described above, some improvements or modifications may be made to the present invention without departing from the gist of the present invention.

### EXPLANATION OF REFERENCES

10: ultrasonography system
12: ultrasonic endoscope
14: ultrasonic processor device
16: endoscope processor device
18: light source device
20: monitor
22: water supply tank
24: suction pump
26: insertion part
28: operation part
30: universal cord
32: air and water supply button
34: suction button
36: angle knob
37: treatment tool insertion port
38: connector
40: connector
42: connector
44: air and water supply tube
46: suction tube
48: endoscope observation portion
50: ultrasonic transducer
52: distal end rigid portion
52A: proximal end
52B: distal end
54: bendable portion
56: soft portion
58: bending piece
59: distal end bending piece
60: bending operation wire
62: ultrasound oscillator
64: distal end side cap
64A: distal end
65: distal end part body
66: exterior constituent member
68: frame
70: observation system unit
72: shielded cable
74: forceps conduit
76: distal end surface
78: treatment tool outlet port
80: observation window
82: illumination window
84: nozzle
86: objective lens
88: prism
90: imaging element
92: substrate
94: signal cable
95: lens barrel
96: forceps pipe
98: forceps tube
100: balloon
102: mounting groove
104: mounting groove
106: supply port
110: band portion
112: band portion
120: annular connecting portion
120A: proximal end
122: annular connecting portion
122A: proximal end
124: flange portion
126: screw
128: hole portion
130: screw
132: hole portion
134: hole portion
136: hole forming portion
136A: semi-circular portion
138: angle rubber
200: distal end rigid portion
300: distal end rigid portion
302: hole forming portion
400: distal end rigid portion
500: distal end rigid portion
A: longitudinal axis
B: peripheral direction
C: tangent line
D: distance
E: distance
L1: rigid length
L2: length of distal end part
L3: length of distal end part
L4: rigid length
L5: rigid length
L6: length of distal end part
L7: rigid length
L8: rigid length

What is claimed is:

1. An endoscope comprising:
a distal end rigid portion that constitutes a distal end side of an endoscope insertion part;
a bendable portion that is bendable and is consecutively provided on a proximal end side of the distal end rigid portion;
an exterior constituent member that constitutes an exterior of the distal end rigid portion;
an internal constituent member disposed inside the exterior constituent member;
a bendable portion constituent member that constitutes a connecting part with the exterior constituent member;
a first fixture that fixes the exterior constituent member and the internal constituent member to each other; and
a second fixture that fixes the exterior constituent member and the bendable portion constituent member to each other,
wherein the first fixture and the second fixture are disposed at positions different from each other in a peripheral direction around a longitudinal axis direction of the endoscope insertion part, and
the first fixture and the second fixture are disposed close to each other in the longitudinal axis direction relative to a case where the first fixture and the second fixture are disposed at the same position in the peripheral direction and are disposed at positions where a distance in the longitudinal axis direction between the first fixture and the second fixture is shortest.

2. The endoscope according to claim 1,
wherein the exterior constituent member has a first hole portion into which the first fixture is inserted and a second hole portion into which the second fixture is inserted,
the first hole portion and the second hole portion are disposed at positions different from each other in the peripheral direction, and
the first hole portion and the second hole portion are disposed close to each other in the longitudinal axis direction relative to a case where the first hole portion and the second hole portion are disposed at the same position in the peripheral direction and are disposed at positions where a distance in the longitudinal axis direction between the first hole portion and the second hole portion is shortest.

3. The endoscope according to claim 2,
wherein the exterior constituent member has a hole forming portion that forms one of the first hole portion and the second hole portion, and at least a part of the hole forming portion is disposed at a position that overlaps with the other of the first hole portion and the second hole portion in the longitudinal axis direction.

4. The endoscope according to claim 2,
wherein the first hole portion and the second hole portion are disposed at positions that at least partially overlap with each other in the longitudinal axis direction.

5. The endoscope according to claim 2,
wherein a distance in the longitudinal axis direction between centers of the first hole portion and the second hole portion is shorter than a sum of a radius of the first hole portion and a radius of the second hole portion.

6. The endoscope according to claim 2,
wherein the exterior constituent member is provided with two first hole portions and two second hole portions.

7. The endoscope according to claim 2,
wherein the exterior constituent member is provided with three first hole portions and three second hole portions.

8. The endoscope according to claim 2,
wherein the first hole portion and the second hole portion are screw holes.

9. The endoscope according to claim 8,
wherein the first fixture and the second fixture are screw members.

10. The endoscope according to claim 1,
wherein the internal constituent member is an ultrasound oscillator fixing frame having an outer peripheral surface on which a plurality of ultrasound oscillators are arranged along the peripheral direction.
